(19) United States
(12) Patent Application Publication
(10) Pub. No.: US 2011/0201047 A1
(43) Pub. Date: Aug. 18, 2011

(54) NOVEL SORTING TECHNOLOGY THAT ALLOWS FOR HIGHLY EFFICIENT SELECTION OF SPERM WITHOUT CHROMATIN DAMAGE

(75) Inventor: Darius A. Paduch, Jersey City, NJ (US)

(73) Assignee: CORNELL UNIVERSITY, Ithaca, NY (US)

(21) Appl. No.: 13/124,246

(22) PCT Filed: Oct. 14, 2009
(86) PCT No.: PCT/US09/60636

§ 371 Date: Apr. 14, 2011

Related U.S. Application Data
(60) Provisional application No. 61/105,223, filed on Oct. 14, 2008.

(51) Int. Cl. C12Q 1/02

(52) U.S. Cl. 435/29

(57) ABSTRACT

This invention provides a method to select and enrich healthy sperm for use in assisted reproductive techniques comprising incubating sperm with at least one marking reagent that enters unhealthy sperm but not healthy sperm. The invention also provides a method to sort healthy sperm of high viability from apoptotic, necrotic and dead sperm by using YO-PRO-1 and PI for fluorescence activated cell sorting in flow cytometers. A kit comprising YO-PRO-1 and PI is also included in this invention.

### BACKGROUND

Most in vitro fertilization (IVF) failures result from male factor deficiencies. The quality of sperm is one of the factors determining the success rate of IVF. The use of randomly selected sperm for this procedure can result in various anomalies of sperm decondensation and embryonic development. Currently, sperm quality is evaluated by conventional semen analysis using a light microscope to determine sperm concentration, motility and morphology. However, conventional semen analysis has limited clinical value for predicting the success rate of IVF as 50% of couples with failed fertilization have normal pre-IVF semen analysis. Furthermore, conventional semen analysis does not assess the presence of apoptotic spermatozoa, which might be partially responsible for the low fertilization and implantation rates in assisted reproduction. Sperm chromatin damage may be a negative predictor for IVF outcomes in couples with recurrent spontaneous abortions and poor embryo development. Methods for detecting chromatin damage such as SCSA, TUNEL, Halosperm and Comet assays require permanent fixation of sperm, making the them unusable for in vitro fertilization or agricultural insemination for livestock and pets. Early apoptotic events resulting in chromatin damage are usually accompanied by increased permeability of the cell membrane to large ions. In this invention, the use of marking reagent is disclosed for fluorescence activated cell sorting to identify apoptotic sperm with chromatin damage, and sort out healthy sperm for use in assisted reproductive technology.

### SUMMARY

This invention provides a method to select and enrich healthy sperms for use in assisted reproduction techniques comprising: a) incubating sperms with at least one marking reagent that enters unhealthy sperm cells but not healthy sperm cells; b) sorting marked sperm cells from unmarked sperm cells. The unhealthy cells can be apoptotic, necrotic, or dead. The marking reagents that may be used here include fluorescent dyes such as YO-PRO-1 and propidium iodide (PI). A flow cytometer can be used to sort marked sperm from unmarked sperm. A kit is provided, comprising YO-PRO-1 and propidium iodide (PI).

### BRIEF DESCRIPTION OF THE FIGURES

FIG. 1: Semen samples from 18 men were analyzed with propidium iodide (PI) and YO-PRO-1. Dead sperm are permeable to PI, and apoptotic sperm are permeable to YO-PRO-1. Intact cells are impermeable to both. Hoechst 33342 was used to calibrate the BD LSR II flow cytometer. Semen samples from five patients are sorted with the BD FACS Vantage. Healthy sperm were separated from necrotic and apoptotic cells based on fluorochrome staining.

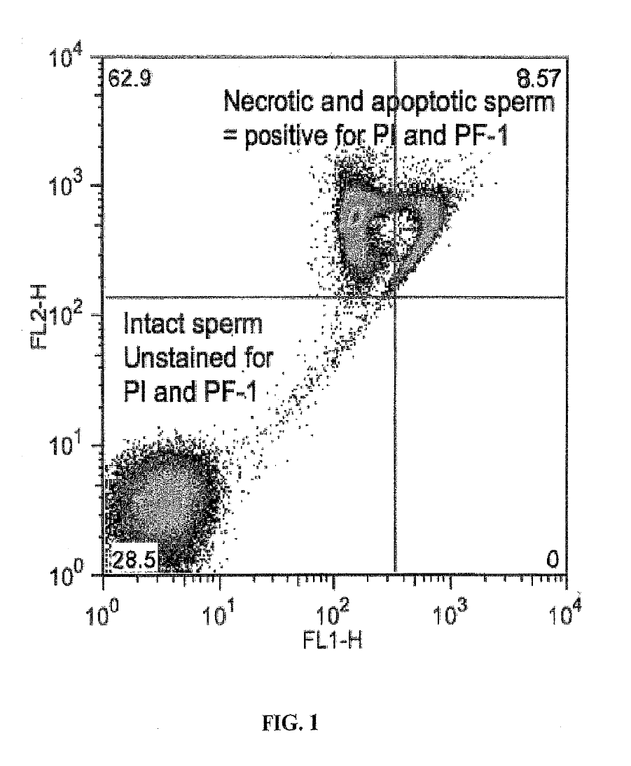

### DETAILED DESCRIPTION

This invention involves the use of marking reagents to label unhealthy sperm cells. In particular, apoptotic cells are permeable by YO-PRO-1, while healthy sperms are not. Necrotic and dead sperms are permeable by propidium iodide and healthy sperms are not. The unhealthy sperm cells that have absorbed the dye are identifiable by the fluorescence of the dye and can be separated by known cell-sorting procedures. When YO-PRO-1 is used in conjunction with propidium iodide, a well known fluorescent marker of dead cells, both apoptotic and dead cells can be identified and removed from the sperm sample prior to its use in assisted reproductive techniques, improving the possibility of successful fertilization, implantation, gestation, and birth of healthy offspring.

The method of the present invention permits reduction of unhealthy sperms in a semen sample, hence enrichment of healthy sperms in the same sample. By “reduction of unhealthy sperms”, it is meant that the number of unhealthy sperms in a sperm sample or population is reduced by at least two (2) fold, at least 3 fold, at least 3 fold, at least 5 fold, at least 6 fold, or at least 7 fold or more.

The advantage of this approach is that the integrity of healthy sperms in the sample is preserved, so that the remaining healthy sperm cells can still be used in assisted reproductive techniques.

The method may be used with human sperms, or with sperms from animals such as bulls, stallions, and dogs.

### DEFINITIONS

### Healthy Sperm

Healthy sperms refers to sperms that have normal morphology and motility, are not apoptotic, necrotic, or dead, and are able to produce a fertilized egg naturally or when introduced to an egg using assisted reproductive techniques.

### Apoptosis

Apoptosis refers broadly to any “programmed cell death” in which single or groups of cells that are part of a multicellular organism die in a regulated process. Broadly speaking necrosis is the other form of cell death in a multicellular organism, and is the process by which cells die under stress, disease, or other attack.

The characteristic morphology of apoptotic cells includes increases in the permeability of the plasma membrane to water and large ions, loss of membrane asymmetry and attachment, formation of irregular bulges in the cell membrane caused by localized decoupling of the cytoskeleton from the plasma membrane, cell shrinkage, nuclear fragmentation, chromatin condensation, and chromosomal DNA fragmentation. Apoptosis is different from necrosis, as the processes associated with apoptosis in disposal of cellular debris do not damage the organism.

### Apoptotic Sperm

Apoptotic sperms are sperms that are going through apoptosis. An early apoptotic event in cells is characterized by increases in the permeability of plasma membranes and loss of phospholipid asymmetry. Since successful fertilization requires a sperm plasma membrane with normal integrity and function, apoptotic sperm will have very low fertility.

### Necrosis

Necrosis refers to the unnatural death of cells and living tissue. It begins with cell swelling, chromatin digestion, and disruption of the plasma membrane and organelle membranes. Late necrosis is characterized by extensive DNA hydrolysis, vacuolation of the endoplasmic reticulum, organelle breakdown, and cell lysis. In contrast to apoptosis, cleanup of cell debris by phagocytes of the immune system is generally more difficult, as the disorderly death generally does not send signals to nearby phagocytes to engulf the dying cell. This lack of signaling makes it harder for the immune system to locate and recycle dead cells which have died through necrosis than if the cell had undergone apoptosis.

### Assisted Reproductive Techniques

Assisted reproductive techniques (ART) is a general term referring to methods used to achieve pregnancy by artificial or partially artificial means. In general, ARTs involve surgically removing eggs from a female, obtaining sperm from a male, combining the egg with sperm in the laboratory, and at some point returning the fertilized egg or embryo to a female body for gestation. ART also include treatments in which only sperm are handled (i.e., artificial insemination) or procedures in which a woman takes medicine only to stimulate egg production without the intention of having eggs retrieved.

When ARTs are used in non-human animals, the process is generally called artificial insemination.

Examples of ARTs include, but are not limited to:

In vitro Fertilization

In vitro fertilization (IVF) is the technique of letting fertilization of the male and female gametes (sperm and egg) occur outside the female body.

Transvaginal Ovum Retrieval (OCR)

This is the process whereby a small needle is inserted through the back of the vagina and guided via ultrasound into the ovarian follicles to collect the fluid that contains the eggs.

Assisted Zona Hatching (AZH)

Assisted zona hatching is performed shortly before the embryo is transferred to the uterus. A small opening is made in the outer layer surrounding the egg in order to help the embryo hatch out and aid in the implantation process of the growing embryo.

Intracytoplasmic Sperm Injection (ICSI)

Intracytoplasmic sperm injection is beneficial in the case of male factor infertility where sperm counts are very low or failed fertilization occurred with previous IVF attempt(s). The ICSI procedure involves a single sperm carefully injected into the center of an egg using a microneedle.

Autologous Endometrial Coculture

Autologous endometrial coculture is a possible treatment for patients who have failed previous IVF attempts or who have poor embryo quality. The patient's fertilized eggs are placed on top of a layer of cells from the patient's own uterine lining, creating a more natural environment for embryo development.

Zygote Intrafallopian Transfer (ZIFT)

In zygote intrafallopian transfer egg cells are removed from the woman's ovaries and fertilized in the laboratory; the resulting zygote is then placed into the fallopian tube.

Egg Donor

Egg donors are resources for women with no eggs due to surgery, chemotherapy, or genetic causes; or with poor egg quality, previously unsuccessful IVF cycles or advanced maternal age. In the egg donor process, eggs are retrieved from a donor's ovaries, fertilized in the laboratory with the sperm from the recipient's partner, and the resulting healthy embryos are returned to the recipient's uterus.

Gestational Carrier

A gestational carrier is an option when a patient's medical condition prevents a safe pregnancy, when a patient has ovaries but no uterus due to congenital absence or previous surgical removal, and where a patient has no ovaries and is also unable to carry a pregnancy to full term.

Cryopreservation

Eggs, sperm and reproductive tissue can be preserved for later IVF.

The following Assisted Reproduction techniques don't necessarily involve IVF.

Gamete intrafallopian transfer (GIFT)

In gamete intrafallopian transfer a mixture of sperm and eggs is placed directly into a woman's fallopian tubes using laparoscopy following a transvaginal ovum retrieval.

Preimplantation Genetic Diagnosis (PGD)

PGD involves the use of Fluorescent In Situ Hybridization (FISH) or Polymerase Chain Reaction (PCR) DNA amplification to help identify genetically abnormal embryos and improve healthy outcomes.

Sex Selection

Sex selection is the attempt to control the sex of offspring to achieve a desired sex. It can be accomplished in several ways, both pre- and post-implantation of an embryo, as well as at birth. Pre-implantation techniques include POD, but also sperm sorting.

Artificial Insemination

Artificial insemination (AI) is when sperm is placed into a female's uterus (intrauterine) or cervix (intracervical) using artificial means rather than by natural copulation.

Therapeutic Donor Insemination

Therapeutic donor is an expansion of artificial insemination. It is also called artificial insemination by donor and is used in situations where the woman doesn't have a partner with functional sperm. Instead, a sperm donor supplies the sperm.

Surgical Sperm Retrieval (SSR)

The reproductive urologist may obtain sperm from the vas deferens, epididymis or directly from the testis in a short outpatient procedure.

Frozen Embryo Transfer (FET)

A fertilized embryo can be cryopreserved. The latter insertion in the body is by the technique Frozen Embryo Transfer (FET).

Marking Reagents

The term marking reagent refers to any kind of label or reagent that can give a readable signal indicating the existence of a molecule, the presence (past or present) of living organisms, the physiological state of living cells, disease conditions, viral infections or a physical process.

For example, marking reagents may also be used to mark cells that are dead or undergoing necrosis.

Reagents that mark cells that are dead or undergoing necrosis include, but are not limited to: propidium iodide.

For example, marking reagents may be used to mark cells that are undergoing apoptosis.

Marking reagents for apoptotic cells include, but are not limited to:

Reagents that only enter and stain cells that are apoptotic, and will not enter healthy cells. For example, YO-PRO-1.

Reagent that mainly enters cells that apoptotic and shows strong staining, but only enters healthy cells in minor amounts and stains them dimly. For example, Hoechst 33342.

Reagents that will only bind to certain cell surface molecules of apoptotic cells, but not healthy cells.

Reagent that will only bind to certain cell surface molecules of apoptotic cells include, but are not limited to: Annexin V, which bind to phosphatidylserine that are redistributed from the inner plasma membrane leaflet to the out leaflet during the early onset of apopotisis. When. Annexin V is conjugated with fluorescent chemicals or magnetic beads, it can give out readable signals for identification of apoptotic cells.

Reagents that bind to overexpressed key proteins of apoptosis-related pathways. Reagents that bind to overexpressed key proteins of apoptosis-related pathways include, but are not limited to: antibodies against Cytochrome c, cleaved Caspase-3, cleaved PARP (poly-ADP-ribose-polymerase), Fas, Bcl-x, and p53. When these antibodies are conjugated with fluorescent chemicals or magnetic beads, they can give out readable signals for identification of apoptotic cells.

### Methods to Separate Cells

Several methods to separate cells that are marked from cells that are unmarked are known in the art. The term “marked cells” refers to cells which a marking reagent has bound to and in some instances penetrated into.

Separation methods include, but are not limited to:

Fluorescence-activated cell sorting (FACS), Magnetic-activated cell sorting (MACS), density gradient cell sorting, selection of desired cells by sedimentation, affinity adsorption or affinity extraction.

### EXAMPLES

Semen samples from 18 men were analyzed using propidium iodide (PI) and YO-PRO-1 (PF-1). Dead sperm are permeable to PI, and apoptotic cells are permeable to PF-1. Intact cells are impermeable to both. Hoechst 33342 was used to calibrate the BD LSR II flow cytometer. Semen samples from five patients were sorted with the BD FACSVantage. Normal spermatozoa were separated from necrotic and apoptotie cells based on fluorochrome staining. Percoll density gradient was used to remove debris prior to sorting. To verify selection of intact sperm, the stained and unstained populations were examined microscopically for motility and viability, and analyzed for DNA fragmentation with the TUNEL assay. The two populations were compared using the chi-square test for difference in the percentage of TUNEL-positive cells.

RESULTS: In the group positive for PF-1 and PI, 431 of 2,167 sperm (19.5%) were TUNEL positive. In the non-staining group only 33 of 2,263 (1.5%) fluoresced. The difference was highly statistically significant (p<0.00001). Unstained sperm had excellent progressive motility and normal morphology.

This technology allows for sperm chromatin damage analysis as well as quick and reliable sorting, separating normal sperm from those with chromatin damage. Because the test employs large molecules that require activation of sodium channels to enter sperm, there is little risk for residual fluorochrome in the isolated specimen. This assay may be a new treatment modality for couples with male factor infertility secondary to sperm chromatin damage.

The following statements are potential claims that may be converted to claims in a future application. No modifications of the following statements should be allowed to affect the interpretation of claims which may be drafted when this provisional application is converted into a regular utility application.

1. A method to select healthy sperm for use in assisted reproductive technologies comprising:
a) incubating sperm with at least one marking reagent that enters unhealthy sperm cells but not healthy sperm cells, and b) separating marked sperm from unmarked sperm.

2. A method according to claim 1, wherein the unhealthy sperm are apoptotic.

3. A. method according to claim 1 wherein the unhealthy sperm are necrotic.

4. A method according to claim 1, wherein the reagent fluoresces.

5. A method according to claim 1, wherein the fluorescent reagent is YO-PRO-1.

6. A method according to claim 1, wherein the fluorescent reagent is propidium iodide.

7. A method according to claim 1, wherein sperms are incubated with both YO-PRO-1 and propidium iodide prior to the separating step.

8. A method according to claim 1, wherein the sperm is sorted using a flow cytometer.

9. A method of reducing unhealthy sperms in a semen sample for use in assisted reproductive technologies comprising:
a) incubating said semen sample with at least one marking reagent that enters unhealthy sperm cells but not healthy sperm cells in said sample, and
b) separating marked sperms from unmarked sperm, thereby reducing unhealthy sperms in said sample.

10. The method of claim 9, wherein the amount of unhealthy sperms in said sample is reduced by at least two to seven fold.

11. A kit comprising YO-PRO-1 and propidium iodide
